(19) United States
(12) Patent Application Publication
(10) Pub. No.: US 2012/0232142 A1
(43) Pub. Date: Sep. 13, 2012

(54) NOVEL USE OF DIMETHYLFUMARATE

(75) Inventor: In Kyu LEE, Daegu-si (KR)

(73) Assignee: KYUNGPOOK NATIONAL UNIVERSITY INDUSTRY ACADEMIC COOPERATION FOUNDATION, Daegu-si (KR)

(21) Appl. No.: 13/410,020

(22) Filed: Mar. 1, 2012

Related U.S. Application Data
(63) Continuation of application No. 12/677,089, filed on Mar. 8, 2010.

(30) Foreign Application Priority Data
Sep. 13, 2007 (KR) 10-2007-0093309

(51) Int. Cl. A61K 31/225

(52) U.S. Cl. 514/547

(57) ABSTRACT

Disclosed are a pharmaceutical composition for inhibiting vascular smooth muscle cell proliferation comprising dimethyl fumarate as an effective ingredient, use of dimethyl fumarate for inhibiting vascular smooth muscle cell proliferation, and a method of inhibiting vascular smooth muscle cell proliferation employing the same. Through the present invention, it was found that dimethyl fumarate could inhibit vascular smooth muscle cell proliferation by increasing the activity of AMPK. Accordingly, dimethyl fumarate can be usefully used as an effective ingredient of a medicine for inhibiting vascular smooth muscle cell proliferation.

### TECHNICAL FIELD

The present invention relates to a pharmaceutical composition for inhibiting vascular smooth muscle cell proliferation comprising dimethyl fumarate as an effective ingredient, use of dimethyl fumarate for inhibiting vascular smooth muscle cell proliferation, and a method of inhibiting vascular smooth muscle cell proliferation employing the same.

### BACKGROUND ART

Vascular smooth muscle cell proliferation is a crucial cause of a cardiovascular disease including arteriosclerosis such as atherosclerosis and vascular restenosis (Hidde B., Restenosis: a challenge for pharmacology. Trends. Pharmacol. Sci. 2000; 21(7):274-279; Nageswara R M, and Marschall S R, Circ. Res. 2007; 100:460-473; Andres V, Castro C. Antiproliferative strategies for the treatment of vascular proliferative disease. Curr Vasc Pharmacol. 2003 March; 1(1):85-98; Hao H, Gabbiani G, Bochaton-Piallat M L. Arterial smooth muscle cell heterogeneity: implications for atherosclerosis and restenosis development. Arterioscler Thromb Vasc Biol. 2003 Sep. 1; 23(9):1510-20).

The best strategy for preventing such cardiovascular disease is to control the factor of a metabolic syndrome such as hypertension, hyperlipidemia, obesity and diabetes mellitus well. However, once such disease is attacked, a therapy using a drug or an operational method is required. Blood pressure is controlled by a statin-based drug and an antihypertension drug. However, such drug alleviates only about 15 to 30% of the cardiovascular disease, and thus it cannot be a radical therapy. The best therapy known hereto is a method that a catheter having a balloon is inserted into a blocked or narrowed blood vessel thereby opening the blood vessel through dilating the balloon (Hidde B., Restenosis: a challenge for pharmacology. Trends. Pharmacol. Sci. 2000; 21(7):274-279; Andres V, Castro C. Antiproliferative strategies for the treatment of vascular proliferative disease. Curr Vasc Pharmacol. 2003 March; 1(1):85-98; Hao H, Gabbiani G, Bochaton-Piallat M L. Arterial smooth muscle cell heterogeneity: implications for atherosclerosis and restenosis development. Arterioscler Thromb Vasc Biol. 2003 Sep. 1; 23(9):1510-20). However, there is a problem that about 50% of restenosis occur within about one year after balloon dilation due to vascular smooth muscle cell re-proliferation, and thus it is necessary to inhibit vascular smooth muscle cell proliferation.

Recently, researches linking various metabolic diseases with mitochondria are actively progressed. Oxidation stress is increasingly observed in vascular cells during pathogenesis of vascular complications, and there is a ruling opinion that such increase of oxidation stress results from malfunction of mitochondria (Nageswara R M and Marschall S R, Circ. Res. 2007; 100:460-473). That is because mitochondria is an organelle that generates active oxygen species in association with glucose metabolism and fat metabolism in various systems of generating oxidation stress, and commonly acts on oxidation stress generated by high glucose level in blood, a fatty acid, a cytokine and a growth factor, etc., thereby further accelerating occurrence of vascular complications. In recent research, over-expression of genes such as UCP-2, AMPK and PGC-1 was observed to improve the function of mitochondria and inhibit proliferation and migration of a vascular smooth muscle cell by a hypertension inducing agent (Lee W. J., et al., Arterioscler Thromb Vasc Biol. 2005; 25:2488-2494; Park J. Y., et al., Diabetologia 2005; 48:1022-1028; Lee I K, et al., Effects of Recombinant Adenovirus-Mediated Uncoupling Protein 2 Overexpression on Endothelial Function and Apoptosis. Circ Res. 2005 Jun. 10; 96(11):1200-7; Kim H J, et al., Effects of PGC-1α on TNF-α Induced MCP-1 and VCAM-1 Expression and NF-κ B Activation in Human Aortic Smooth Muscle and Endothelial Cells. ANTIOXIDANTS & REDOX SIGNALING. 2007; 9(3): 301-307).

It was again reported that vascular smooth muscle cell proliferation could be under the control of the activity of AMPK (Nagata D, et al., AMP-activated protein kinase inhibits Angiotensin II-stimulated vascular smooth muscle cell proliferation. Circulation: 2004; 110:444-451). It was observed that the proliferation of a vascular smooth muscle cell in which AMPK was activated was inhibited, and the expression of p53 and p21, which are cell proliferation inhibitors, was increased and the activity of CDK (cyclin-dependent kinase) was decreased in such a vascular smooth muscle cell (Igata M, et al., Adenosine monophosphate-activated protein kinase suppresses vascular smooth muscle cell proliferation through the inhibition of cell cycle progression. Circ Res. 2005; 97(8):837-844). AMPK is a kind of phosphorylase to be activated when relative percentage of AMP is higher than that of ATP by diet and exercise, and is a crucial protein involved in a metabolism having a function of inhibiting further consumption of ATP by stopping replication of a cell (Hardie D G. AMP-activated protein kinase as a drug target. Annu. Rev. Pharmacol. Toxicol. 2007; 47:185-210). It is known that activated AMPK accelerates glucose metabolism and lipid metabolism, and inhibits glucogenesis and lipid synthesis. In addition, AMPK is also activated regardless of metabolic process, and for example, is also activated by meformin known as a therapeutic agent of diabetes mellitus, and alpha-lipoic acid (Lee W. J., et al., Arterioscler Thromb Vasc Biol. 2005; 25:2488-2494; Lee K M, et al., Alpha-lipoic acid inhibits fractalkine expression and prevents neointimal hyperplasia after balloon injury in rat carotid artery. Atherosclerosis. 2006 November; 189(1): 104-14).

The present inventors completed the present invention by confirming, through research on the material that promotes the activity of AMPK in the vascular smooth muscle cell, that dimethyl fumarate (DMF) promotes the activity of AMPK in the vascular smooth muscle cell thereby inhibiting vascular smooth muscle cell proliferation.

### DISCLOSURE

### Technical Problem

Accordingly, the object of the present invention is to provide a pharmaceutical composition for inhibiting vascular smooth muscle cell proliferation comprising dimethyl fumarate as an effective ingredient, use of dimethyl fumarate for inhibiting vascular smooth muscle cell proliferation, and a method of inhibiting vascular smooth muscle cell proliferation employing the same.

### Technical Solution

The present invention provides a pharmaceutical composition for inhibiting vascular smooth muscle cell proliferation comprising dimethyl fumarate as an effective ingredient.

Dimethyl fumarate has a structure of formula 1 below:

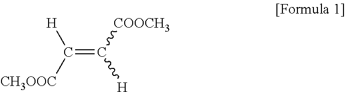

According to an embodiment of the present invention, dimethyl fumarate inhibits vascular smooth muscle cell proliferation, and also decreases the formation of neointima to be generated after balloon dilation. As can be ascertained in the examples below, dimethyl fumarate activates AMPK thereby inhibiting vascular smooth muscle cell proliferation, and further increases the expression of p53 and p21 proteins involved in inhibiting cell proliferation, and inhibits the expression of CDK involved in inducing cell proliferation.

A protein that plays key roles in induction to mitotic stage in cell proliferation and transcriptional activity is E2F. E2F is present in the form bound with a retinoblastoma (Rb), and when Rb is phosphorylated by a growth factor or CDK stimulus, E2F is separated thereby inducing a cell to replication phase. According to Examples below, the phosphorylation of Rb is inhibited in a vascular smooth muscle cell that has reacted with dimethyl fumarate. Accordingly, it can be confirmed through the present invention that dimethyl fumarate has a function of controlling cell cycle.

A composition comprising dimethyl fumarate according to the present invention as an effective ingredient can be prepared by using a pharmaceutically suitable and physiologically acceptable adjuvant in addition to the effective ingredient. As an adjuvant, a solubilizer such as an excipient, a disintegrant, a sweetener, a binder, a coating agent, a blowing agent, a lubricant, a slip modifier or a flavoring agent can be used.

A composition comprising dimethyl fumarate according to the present invention as an effective ingredient can be preferably formulated into a pharmaceutical composition by further comprising at least one pharmaceutically acceptable carrier in addition to the effective ingredient for administration.

A pharmaceutical preparation form of a composition comprising dimethyl fumarate according to the present invention as an effective ingredient may be a granule, a powder, a tablet, a coated tablet, a capsule, a suppository, an enema, a syrup, a juice, a suspension, an emulsion or an injectable liquid, etc.

For example, for formulation into a form of a tablet or a capsule, the effective ingredient can be combined with an oral, non-toxic, pharmaceutically acceptable, inert carrier such as ethanol, glycerol and water, etc. In addition, if desired or required, a suitable binder, a lubricant, a disintegrant and a coloring agent can be also included into a mixture. Examples of the suitable binder include, but are not limited to, starch, gelatin, a natural sugar such as glucose or β-lactose, a corn sweetener, acacia, a natural and synthetic gum such as tragacanth or sodium oleate, sodium stearate, magnesium stearate, sodium benzoate, sodium acetate, sodium chloride, etc. Examples of the disintegrant include, but are not limited to, starch, methyl cellulose, agar, bentonite, xanthan gum, etc.

As the pharmaceutically acceptable carrier in a composition formulated into a liquid solution, which is sterilized and suitable to a living body, saline water, sterilized water, a linger solution, buffered saline water, an albumin injection, a dextrose solution, a malto dextrose solution, glycerol, ethanol and a mixture of at least one ingredients thereof can be used, and if needed, other usual additives such as an anti-oxidizing agent, a buffer solution and a bacteriostatic agent can be added. Further, an injectable formulation such as an aqueous solution, a suspension and an emulsion, a pill, a granule or a tablet can be formulated by further adding a diluent, a dispersant, a surfactant, a binder and a lubricant. Furthermore, formulation can be preferably achieved according to each disease or an ingredient by employing a method disclosed in Remington's Pharmaceutical Science, Mack Publishing Company, Easton Pa. as a proper method in the art.

The present invention also provides a use of dimethyl fumarate for preparing a medicine for inhibiting vascular smooth muscle cell proliferation.

The pharmaceutical composition for inhibiting vascular smooth muscle cell proliferation can be used for preparing such a medicine.

Further, the present invention provides a method of inhibiting vascular smooth muscle cell proliferation comprising administering a pharmaceutical composition comprising therapeutically effective amount of dimethyl fumarate as an effective ingredient to a mammal.

According to the present invention, inhibiting vascular smooth muscle cell proliferation includes decreasing and preventing vascular smooth muscle cell proliferation.

The pharmaceutical composition for inhibiting vascular smooth muscle cell proliferation according to the present invention can be used for preventing or treating cardiovascular diseases including arteriosclerosis (Hidde B., Restenosis: a challenge for pharmacology. Trends. Pharmacol. Sci. 2000; 21(7):274-279; Nageswara R M, and Marschall S R, Circ. Res. 2007; 100:460-473; Andres V, Castro C. Antiproliferative strategies for the treatment of vascular proliferative disease. Curr Vasc Pharmacol. 2003 March; 1(1):85-98; Hidde B., Restenosis: a challenge for pharmacology. Trends. Pharmacol. Sci. 2000; 21(7):274-279; Hao H, Gabbiani G, Bochaton-Piallat M L. Arterial smooth muscle cell heterogeneity: implications for atherosclerosis and restenosis development. Arterioscler Thromb Vasc Biol. 2003 Sep. 1; 23(9):1510-20) such as atherosclerosis that is a disease caused by vascular smooth muscle cell proliferation.

Accordingly, the pharmaceutical composition for inhibiting vascular smooth muscle cell proliferation according to the present invention can also comprise one or more therapeutic agents for treating cardiovascular diseases. For example, dimethyl fumarate can be used together with a therapeutic agent for treating hyperlipidemia or a hypotensive agent well known to those skilled in the art.

A composition comprising dimethyl fumarate according to the present invention as an effective ingredient can be administered in usual ways via intravenous, intra-arterial, peritoneal, intramuscular, intrasternal, transdermal, nasal, inhalation, topical, rectal, oral, intraocular or intracutaneous route.

Therapeutically effective amount of the composition comprising dimethyl fumarate according to the present invention as an effective ingredient refers to an amount required in achieving an effect of inhibiting vascular smooth muscle cell proliferation. Accordingly, the therapeutically effective amount can be controlled according to various factors including type of disease, seriousness of disease, type and content of an effective ingredient and other ingredient contained in the composition, type of a formulation, and age, weight, general health status, sex and diet of a patient, administration time, administration route and secretion rate of the composition, treatment period and a drug used simultaneously. Preferably, dimethyl fumarate can be administered to an adult once to several times daily, for example, in a dose of 100 mg/kg to 1,000 mg/kg.

### Advantageous Effects

Through the present invention, it was found that dimethyl fumarate could inhibit vascular smooth muscle cell proliferation by increasing the activity of AMPK. Accordingly, dimethyl fumarate can be usefully used as an effective ingredient of a medicine for inhibiting vascular smooth muscle cell proliferation.

### DESCRIPTION OF DRAWINGS

FIG. 1 depicts a graph showing that vascular smooth muscle cell proliferation is significantly decreased dependently on the concentration of dimethyl fumarate when dimethyl fumarate is treated in several concentrations with or without PDGF.

FIG. 2 is a microscopic photograph (×100) showing cut section of carotid artery of a rat two weeks after balloon dilation.

FIG. 3 is a western blot photograph showing the effect of dimethyl fumarate on the phosphorylation of AMPK and Acc.

FIG. 4 is a western blot photograph showing the effect of dimethyl fumarate on the expression of p53 and p21 proteins, which are proteins involved in cell proliferation.

FIG. 5 is a western blot photograph showing the effect of dimethyl fumarate on the expression of pRb and CDK.

FIG. 6 depicts the result of analysis for cell cycle employing FACS showing the effect of dimethyl fumarate on cell cycle.

### BEST MODE

The advantages and features of the present invention and a method of achieving the same will be clarified with reference to Examples described below in detail. However, the present invention is not limited to the Examples disclosed below, but will be embodied into various embodiments different from one another. These examples render the present invention to be more completely disclosed and are presented in order to let those skilled in the art know the scope of the present invention, and the scope of the present invention is defined only by the appended claims.

### EXAMPLES

### Isolation and Cultivation of Vascular Smooth Muscle Cells

For culturing vascular smooth muscle cells, vascular smooth muscle cells were isolated from the aorta of Sprague-Dawley rat and were first cultured. Vascular smooth muscle cells were cultured in a culturing apparatus having conditions of 37° C., 5% carbon dioxide until cells are grown up in culture medium containing 20% bovine fetal serum. Cells obtained from the procedure were transferred to a fresh culture dish to culture, and the initial cells subcultured up to 4 to 7 times were used in experiments.

### Example 1

### Confirmation on Inhibition of Vascular Smooth Muscle Cell Proliferation by Dimethyl Fumarate

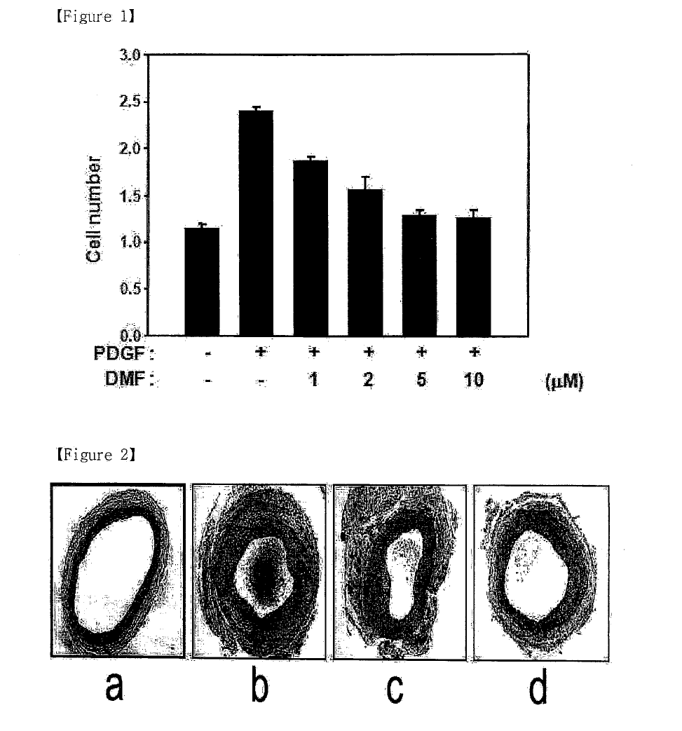

First cultured vascular smooth muscle cells were cultured in 96-well culture dish, and when growth reached 70%, they were transferred to a medium containing 0.5% bovine fetal serum, and then cultured for 24 hours and the cells was stood at interphase status. Different doses (1, 2, 5, 10 μM) of dimethyl fumarate and a platelet derived growth factor (PDGF) (20 ng/ml) that increases cell proliferation were treated in the first cultured vascular smooth muscle cells, and then reaction was performed at 37° C. for 48 hours. The number of viable cells was measured employing WST cell counting kit (WAKO, Japan). A reagent for confirming cell proliferation was treated thereto, reaction was further performed for 4 hours, and then optical density was measured at 450 nm with ELISA reader to investigate the ability of proliferating cells. The results of the experiments were shown by taking an average from values measured in more than three separate experiments. As can be seen in FIG. 1, cell proliferation that was increased by the platelet derived growth factor was inhibited as the concentration of dimethyl fumarate was increased.

### MODE FOR INVENTION

### Example 2

### Confirmation on Inhibition Effect of Vascular Smooth Muscle Cell Proliferation in Rats

In order to confirm whether dimethyl fumarate inhibits the formation of neointima after balloon dilation, experiments were performed with Sprague-Dawley rats fed with food containing dimethyl fumarate.

The rats were bred while maintaining conditions that the temperature of the breeding room was maintained at 22±2° C., and brightness was automatically controlled in 12 hour cycles. Rats were categorized into a normal control group, a negative control group fed with only high fat diet (20% fat, 0.05% cholesterol), and an experiment group fed with food containing 0.5% or 1% dimethyl fumarate together with high fat diet (4 rats per each group), and experiments were progressed while breeding for 4 weeks in separate cages containing one rat per cage. Balloon dilation was performed after breeding rats for 2 weeks before the balloon dilation, and breeding was performed for 2 weeks more while continuing on feeding general diet and dimethyl fumarate diet, and then their aorta were separated and the formation of neointima was confirmed with H & E (hematoxylin and eosin) staining method.

From the results, it was confirmed that neointima were formed in the group (FIG. 2b) fed with only high fat diet compared to the normal control group (FIG. 2a). Meanwhile, it was observed that the formation of neointima after performing balloon dilation was certainly decreased in the groups fed with 0.5% and 1% dimethyl fumarate, respectively (FIG. 2c, FIG. 2d).

### Experimental Example 1

### Confirmation on the Effect of Dimethyl Fumarate on the Phosphorylation of AMPK and ACC

First cultured vascular smooth muscle cells were filled in about 80 to 90% of 60 mm tissue culture dish, and the cells were stood in a medium containing 0.5% FBS for 24 hours to render the cells to be interphase status. The group not treated with dimethyl fumarate was defined as a control, and experimental groups were divided into 5 groups treated with 5 μM dimethyl fumarate for 1, 2, 3, 6, 12 hours, respectively.

Whole proteins were separated from vascular smooth muscle cells of each group by employing RIPA buffer solution (50 mM Tris-HCl, 150 mM NaCl, 5 mM EDTA, 1% NP-40, 1 mM PMSF, 1 mM DTT, 1 mg/ml protease inhibitor). Separated proteins of each sample were quantitatively analyzed, 25 mg of proteins were mixed with sample buffer solution, the mixture was boiled for 5 minutes and then cooled, electrophoresis was performed for the resulting product in sodium dodecyl sulfate polyacrylamide gel to separate depending on size, then the product was transferred to a PVDF membrane, and then reacted with pAMPK or pACC antibody to confirm phosphorylation thereof. In addition, in order to confirm whether a certain amount of proteins were used, the product was reacted with an anti-actin antibody.

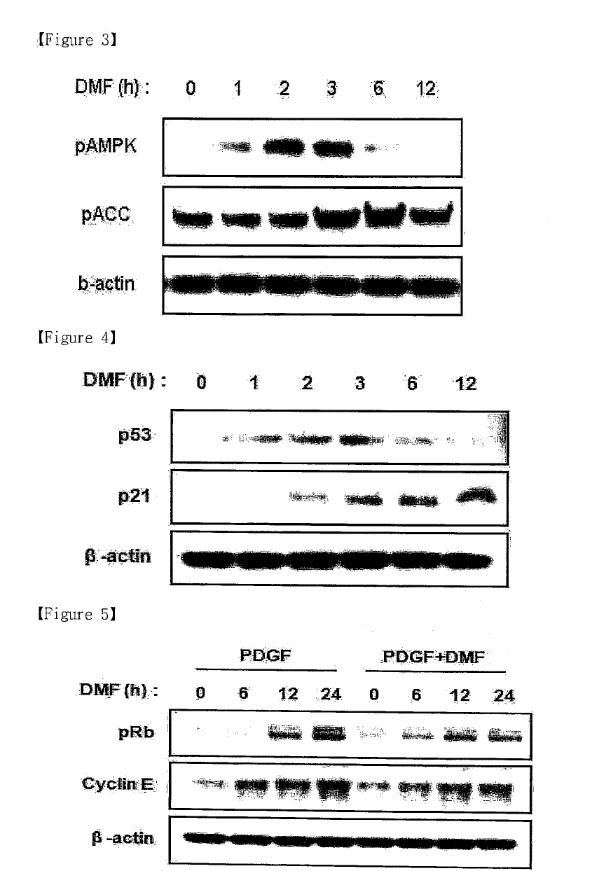

As can be seen in FIG. 3, it was confirmed that a half of the activity of AMPK was initially increased, and the phosphorylation of ACC, which is a target gene of AMPK, was continuously increased.

### Experimental Example 2

### Confirmation on the Effect of Dimethyl Fumarate on the Expression of a Protein Involved in Cell Proliferation

First cultured vascular smooth muscle cells were filled in about 80 to 90% of 60 mm tissue culture dish, and the cells were stood in a medium containing 0.5% FBS for 24 hours to render the cells to be interphase status. The group not treated with dimethyl fumarate was defined as a control, and experimental groups were divided into 5 groups treated with 5 μM dimethyl fumarate for 1, 2, 3, 6, 12 hours, respectively.

Whole proteins were separated from vascular smooth muscle cells of each group by employing RIPA buffer solution (50 mM Tris HCl, 150 mM NaCl, 5 mM EDTA, 1% NP-40, 1 mM PMSF, 1 mM DTT, 1 mg/ml protease inhibitor). Separated proteins of each sample were quantitatively analyzed, 25 μg of proteins were mixed with sample buffer solution, the mixture was boiled for 5 minutes and then cooled, electrophoresis was performed for the resulting product in sodium dodecyl sulfate polyacrylamide gel to separate depending on size, then the product was transferred to a PVDF membrane, and then reacted with p53 or p21 antibody to confirm the expression thereof. In addition, in order to confirm whether a certain amount of proteins were used, the product was reacted with an anti-actin antibody.

From the results, as can be seen in FIG. 4, it was confirmed that expression of p53 and p21, which are proteins involved in cell proliferation, was increased by dimethyl fumarate.

### Experimental Example 3

### Confirmation on the Effect of Dimethyl Fumarate on the Expression of a Protein Involved in Cell Proliferation

First cultured vascular smooth muscle cells were filled in about 80 to 90% of 60 mm tissue culture dish, and the cells were stood in a medium containing 0.5% FBS for 24 hours to render the cells to be interphase status. The group not treated with dimethyl fumarate was defined as a control, and experimental groups were divided into 3 groups treated with 5 μM dimethyl fumarate for 6, 12 and 24 hours, respectively, with or without PDGF. Whole proteins were separated from vascular smooth muscle cells of each group by employing RIPA buffer solution (50 mM Tris-HCl, 150 mM NaCl, 5 mM EDTA, 1% NP-40, 1 mM PMSF, 1 mM DTT, 1 μg/ml protease inhibitor). Separated proteins of each sample were quantitatively analyzed, 25 μg of proteins were mixed with sample buffer solution, the mixture was boiled for 5 minutes and then cooled, electrophoresis was performed for the resulting product in sodium dodecyl sulfate polyacrylamide gel to separate depending on size, then the product was transferred to a PVDF membrane, and then reacted with an antibody against pRb or Cyclin. E to confirm the expression thereof. In addition, in order to confirm whether a certain amount of proteins were used, the product was reacted with an anti-actin antibody.

From the results, as can be seen in FIG. 5, it was confirmed that dimethyl fumarate inhibits the phosphorylation of Rb promoted by a growth factor or CDK (FIG. 5).

Western blotting was performed in order to confirm whether dimethyl fumarate inhibits the expression of CDK. 5 μM dimethyl fumarate were pretreated in the first cultured vascular smooth muscle cells for 2 hours, and PDGF, which is a proliferation factor, was treated thereto. Reaction was performed for a defined period, and then cells were collected and investigated for CDK expression with Western blotting method. From the results, it was confirmed that expression of CDK was more inhibited in the experimental group pretreated with dimethyl fumarate than in the normal control group not treated with dimethyl fumarate (FIG. 5).

### Experimental Example 4

### Confirmation on the Effect of Dimethyl Fumarate on Cell Cycle

Vascular smooth muscle cells were analyzed for cell cycle employing FACS in order to confirm the effect of dimethyl fumarate on cell cycle. 5 μM dimethyl fumarate were pretreated for 2 hours in the vascular smooth muscle cells cultured in a medium containing 0.5% bovine fetal serum for 24 hours. Cells were treated with a growth factor and insulin to induce cells into replication phase, and reaction was performed for 24 hours. Then cells were collected and immobilized, and then their nucleuses were stained with propidium iodide (PI), and cell cycle was analyzed by employing FACS. Cell cycle was indicated as % by measuring 10,000 cells per each sample.

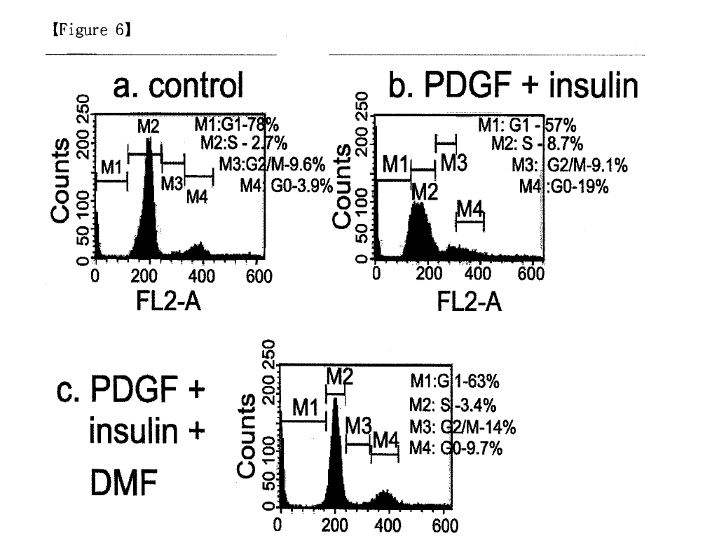

From the results, it was confirmed that cells in replication phase status among vascular smooth muscle cells stimulated with a growth factor and insulin were increased (8.7%) (FIG. 6b) compared to the control group not stimulated (FIG. 6c). Meanwhile, it was confirmed that cells in replication phase status among cells reacted simultaneously with dimethyl fumarate were decreased to 3.4%.

### INDUSTRIAL APPLICABILITY

Through the present invention, it was found that dimethyl fumarate could inhibit vascular smooth muscle cell proliferation by increasing the activity of AMPK. Accordingly, dimethyl fumarate can be usefully used as an effective ingredient of a medicine for inhibiting vascular smooth muscle cell proliferation.

What is claimed is:

1. A method of inhibiting vascular smooth muscle cell proliferation comprising administering a therapeutically effective amount of dimethyl fumarate to a mammal.

2. A method of preventing or treating vascular restenosis comprising administering a therapeutically effective amount of dimethyl fumarate to a mammal.
